(19) United States
(12) Patent Application Publication
(10) Pub. No.: US 2024/0032554 A1
(43) Pub. Date: Feb. 1, 2024

(54) NOVEL PROCESS FOR PREPARING AN ISOLATE OF CATIONIC WHEY PROTEINS AND THE PRODUCT THUS OBTAINED

(71) Applicant: SAVENCIA SA, Viroflay (FR)

(72) Inventors: Takashi MIKOGAMI, Kawasaki Kanagawa (JP); Carine LECHEVIN, Saint Grégoire (FR); Michel BOURTOURAULT, Noyal-Chatillon sur Seiche (FR)

(21) Appl. No.: 18/266,541

(22) PCT Filed: Dec. 22, 2021
(86) PCT No.: PCT/EP2021/087268

§ 371 Date: Jun. 9, 2023

(30) Foreign Application Priority Data
Dec. 22, 2020 (FR) FR2013945

(51) Int. Cl. A23C 9/146; A23C 9/142; A23C 21/00; A23L 33/00; A23L 33/19; A23C 1/04; A23C 1/08

(52) U.S. Cl. CPC A23C 9/1465; A23C 9/1422; A23C 21/00; A23L 33/40; A23L 33/19; A23C 1/04; A23C 1/08

(57) ABSTRACT

The present invention relates to a novel process for preparing an isolate of cationic whey proteins containing high purity lactoferrin.

The present invention relates to a new method for preparing an isolate of cationic whey proteins of high purity lactoferrin.

The applicant has developed a method for obtaining an isolate of whey proteins whose lactoferrin protein purity is higher than 90%; this method allows the control of the vitamin B12 (cobalamin) content in the lactoferrin isolate.

This method is characterised on the one hand by the use of previously concentrated milk material (such as concentrated skimmed milk or concentrated whey) by a membrane technology (such as reverse osmosis, nanofiltration, or ultrafiltration) and on the other hand by a selective extraction using strong cation exchange resins of the sulphopropyl (SP) type packed in a radial chromatography column. The eluted lactoferrin pure fraction is concentrated and demineralised by ultrafiltration to obtain an isolate of cationic whey proteins whose lactoferrin purity higher than at least 90% and preferably 95%. This liquid isolate obtained is debacterised or sterilised by a microfiltration and optionally dried by spray drying or freeze drying to obtain the powder isolate.

The method for preparing an isolate of cationic whey proteins of high purity lactoferrin comprises the following steps a) to f):
a) The starting raw material can be a mammalian milk pre-skimmed and concentrated by a membrane technology; it can also be a mixture of mammalian milk pre-skimmed and concentrated by a membrane technology and skimmed (unconcentrated) milk; the mammalian milk is for example cow's milk or goat's milk; the starting raw material can also be whey from mammalian milk and pre-concentrated;
   i. when the starting raw material is prepared with a mammalian milk such as cow's milk or goat's milk, it is skimmed and optionally pasteurised, for example by a thermal treatment between 60 and 78° C. of short duration (a minimum thermal treatment of equivalent level of 72° C. for 15 seconds; the skimming can be executed either before or after pasteurisation) or debacterised by a microfiltration with a membrane of porosity between and 1.4 μm, and then concentrated by reverse osmosis (RO) or nanofiltration (NF) or ultrafiltration (UF); for the implementation of the method, a mixture of skimmed milk (not concentrated) and skimmed milk previously concentrated by a membrane technology as described above can also be used; the product to be treated in step b) preferably has a concentration of protein material (PM) between 40 and 72 g/L, preferably between 43 and 57 g/L; when an RO membrane is used, the dry material (DM) concentration of pasteurized skimmed milk is between 110 and 200 g/L, preferably between 120 and 160 g/L;
   ii. when the starting raw material is prepared with whey, it can be concentrated after separation of the caseins, by acidification or rennet action, either by microfiltration (whose membrane has a porosity of approximately 0.1 μm), concentrated by reverse osmosis (RO) or nanofiltration (NF) or ultrafiltration (UF); for the implementation of the method, a mixture of whey (not concentrated) and whey previously concentrated by a membrane technology as described above can also be used; the product to be treated in step b) preferably has a protein concentration between 20 and 100 g/L, preferably between 30 and 80 g/L;

It should be noted that the pasteurisation and microfiltration treatments are not essential for the method.
b) selectively extracting cationic proteins comprising the following steps:
   i. passing the starting raw material (e.g. pre-concentrated pasteurised skimmed milk) through a radial flow chromatography column containing strong cation exchange resins of the sulphopropyl SP type, preferably larger than 100 μm in diameter (e.g. SP Sepharose Big Beads from Cytiva Sweden):
      the volume of starting raw material (expressed as a volume equivalent to that of the unconcentrated raw material; i.e. the volume indicated is that which the raw material had before being concentrated) is between 40 and 500 times, in particular between 80 and 500 times the volume of the resins (BV, Bed Volume), preferably between 80 and 300 BV;
      the linear speed of the starting raw material passage is between 1.0 and 4.0 m/h, preferably between 2.0 and 3.0 m/h;
   ii. rinsing with demineralised water, preferably treated with a RO membrane (osmosis water):
      the volume of demineralised water is between 2 and 6 BV, preferably between 3 and 5 BV;
      the demineralised water passage speed is between 3.0 and 5.0 m/h, preferably between 3.5 and 4.5 m/h;
   iii. eluting the bound cationic proteins with a saline solution (NaCl in demineralised water, preferably osmosis water) with an electrical conductivity between 30 and 50 mS/cm:
      the volume of the saline solution is between 4 and 8 BV, preferably between 5 and 7 BV;
      the passage speed of the saline solution is between 0.3 and 2.0 m/h, preferably between 0.5 and 1.0 m/h;
   iv. eluting the bound cationic proteins with a saline solution (NaCl in demineralised water, preferably osmosis water) with an electrical conductivity between 80 and 140 mS/cm, preferably between 90 and 110 mS/cm:
      the volume of the saline solution is between 3 and 6 BV, preferably between 4 and 5 BV;
      the passage speed of the saline solution is between 0.5 and 2.5 m/h, preferably between 1.0 and 2.0 m/h;

The passage step on cation exchange resins serves to bind cationic proteins present in the starting raw material while allowing major constituents of skimmed milk such as lactose, minerals, acidic proteins such as caseins, β-lactoglobulin, α-lactoglobulin, serum albumin and most immunoglobulins to pass. The first elution step serves to selectively extract specific cationic proteins by keeping the majority of lactoferrin, the major protein of milk cationic proteins, bound to the resins. The pure bovine lactoferrin fraction is therefore eluted in the second eluate.
c) concentrating the cationic proteins with high purity lactoferrin eluted in the saline solution using an ultrafiltration membrane with a cutoff threshold (MWCO) between 10 and 20 kDa;
d) demineralizing cationic proteins with high purity lactoferrin by a diafiltration with demineralised water, preferably osmosis water, using an ultrafiltration membrane of MWCO between 10 and 20 kDa to achieve an ash/protein ratio between 0.001 and 0.03, preferably between 0.003 and 0.01;
e) micro-filtrating with a membrane having a cut-off threshold between 0.2 and 1.4 μm, preferably 0.8 and 1.4 μm in double layer, of the concentrated solution of specific cationic proteins in order to reduce the microbial load;
f) optionally, spray drying or freeze drying the concentrated solution of specific cationic proteins previously microfiltered to obtain a powdered lactoferrin isolate.

Advantageously, the use of a mammalian milk material (e.g. pasteurised skimmed cow's milk, cheese whey from pasteurised goat's milk) concentrated by UF/NF/RO membrane allows for a reduction in the flow passage speed for the equivalent amount of protein present for a passage through an extraction column. Due to this extended contact time with the strong cation exchange resins of the SP type, the extraction efficiency of the cationic proteins is significantly improved.

In addition, the use of a radial flow column (e.g. Albert Handtmann Armaturenfabrick GmbH), due to its trapezoidal geometry, allows to sustainably withstand the pressure generated by the passage of a concentrated mammalian milk material through packed resins.

This combination of a concentrated milk material and a radial flow column is essential to execute a stable and regular industrial production.

Another advantage of the method according to the invention is that it can be carried out effectively over a wide temperature range; in particular, while resin manufacturers recommend an implementation at temperatures between 30 and 50° C., the applicant has succeeded in developing a method that is effective at cold temperatures, i.e. at temperatures below 15° C., preferably below 10° C.

The present invention thus relates to an isolate of cationic whey proteins enriched in lactoferrin obtained or obtainable by the method according to the invention, such that the proportion of protein on dry material is greater than or equal to 90% by weight and whose proportion of lactoferrin on the total proteins is greater than 90% by weight, preferably greater than 95% by weight, still more preferably greater than 98% by weight.

The present invention also relates to an isolate of cationic whey proteins enriched with lactoferrin from milk or whey from cow's milk or goat's milk obtained or obtainable by the method according to the invention, whose protein proportion on dry material is greater than or equal to 90% by weight, the proportion of lactoferrin on the total proteins is greater than 95% by weight (w/w), preferably greater than 98% by weight, and containing cobalamin in complex form with transcobalamin at a concentration of less than or equal to 5 μg/g of proteins, in particular, the concentration of cobalamin in complex form with transcobalamin is between 1 and 5 μg/g of proteins.

The present invention also relates to an isolate of cationic whey proteins enriched with lactoferrin from milk or whey from cow's milk or goat's milk obtained or obtainable by the method according to the invention, whose protein proportion on dry material is greater than or equal to 90% by weight, the proportion of lactoferrin on the total proteins is greater than 90% (w/w), preferably greater than 95% by weight, containing cobalamin in complex form with transcobalamin at a concentration greater than or equal to 5 μg/g, preferably greater than or equal to 8 μg/g, still more preferably greater than or equal to 10 μg/g of protein.

The isolates according to the invention may be in liquid form (step f not implemented) or in powder form (step f implemented). If it is in liquid form, it has the same characteristics as the powder in terms of composition relative to the dry material and generally comprises between 5 and 25%, preferably between 10 and 20%, by weight of water.

According to another object, the present invention relates to a food product for human or animal consumption, a human or animal medicine or a food supplement containing an isolate of cationic proteins according to the invention.

Preferably, the isolates according to the invention have a microbial load such that the count of aerobic mesophilic flora is less than 1000, preferably less than 100 or 10 and even more preferably less than 1 cfu/g of powder of isolate according to the invention or less than 100, preferably less than 10, even more preferably less than 1 cfu/ml of liquid isolate. The combination of the use of a concentrated milk material and a radial flow column allows the stable and efficient production of two kinds of isolates of high purity lactoferrin by cation exchange chromatography under the appropriate conditions:
an isolate whose lactoferrin protein purity is >95% or 98% and whose vitamin B12 content is 5 μg/g protein or between 1 and 5 μg/g protein;

Such isolates according to the invention are of particular interest for the preparation of infant formulas (infant milks or follow-on milks) based on cow's or goat's milk.
and
an isolate whose lactoferrin protein purity is >90% or 95% and whose vitamin B12 content is 5 μg/g protein, preferably 8 μg/g protein, more preferably >10 μg/g protein;

This isolate may have a nutritional benefit for a food supplement for vegetarians or for a nutritional preparation for people deficient in the absorption of vitamin B12, such the people who has undergone gastrectomy or people chronically treated with PPI (proton pump inhibitors). Indeed, this isolate can provide, in addition to the benefits of lactoferrin, an important source of vitamin B12 with high bioavailability even in the absence of the intrinsic factor secreted by the stomach.

The present invention thus also relates to dietary supplements comprising the isolate according to the invention enriched in vitamin B12, i.e. an isolate whose lactoferrin protein purity is >90% or 95% and whose vitamin B12 content is 5 μg/g protein, preferably 8 μg/g protein, still more preferably greater than or equal to 10 μg/g protein.

The isolate content according to the invention in the food supplement will be chosen according to the profile of the population to be supplemented, thus the dose of vitamin B12 to be administered, and the vitamin B12 content of the isolate. For example, a daily dose of 150 to 1000 mg protein of the isolate according to the invention enriched with vitamin B12 at 6 μg/g protein can provide 0.9 to 6.0 μg vitamin B12 in complex form with transcobalamin. Also, 100 to 600 mg of protein of the isolate according to the invention enriched with vitamin B12 at 10 μg/g protein can provide 1.0 to 6.0 μg of vitamin B12 in complex form with transcobalamin. Thus, such a food supplement can cover the needs of each population group shown in the table below even if intestinal absorption of vitamin B12 is disturbed.

Nutritional references for vitamin B12 (μg/d) according to ANSES 2016

| Population groups | Adequate intake (μg/day) |
|---|---|
| Infants under 6 months | 0.4 |
| Infants aged 6 months and over | 1.5 |
| Children from 1 to 3 years | 1.5 |
| Children from 4 to 10 years old | 1.5 |
| Adolescents aged 11 to 17 | 2.5 |
| Men and women aged 18 and over | 4 |
| Pregnant women | 4.5 |
| Lactating women | 5 |

The present invention further relates to an isolate whose lactoferrin protein purity is >90% or 95% and whose vitamin B12 content is 5 μg/g protein, preferably 8 μg/g protein, more preferably 10 μg/g protein for preventing and/or treating a vitamin B12 deficient absorption, e.g. in patients who have undergone gastrectomy or chronically treated with PPI (proton pump inhibitors).

Cobalamin (vitamin B12) is present in milk in complex form with a binding protein. In cow's milk, it is present in complex form with transcobalamin, which is a cationic protein of 43 kDa (S. N. Fedosov, T. E. Petersen, E. Nex∅, Transcobalamin from cow milk: isolation and physico-chemical properties, Biochimica et Biophysica Acta—Protein Structure and Molecular Enzymology. 1292 (1996) 113-119). The nutritional interest of this cobalamin-transcobalamin complex is important, as it is thought to be responsible for the bioavailability of vitamin B12 (S. N. Fedosov, Ebba Nexo, Christian W. Heegaard, Vitamin B12 and its binding proteins in milk from cow and buffalo in relation to bioavailability of B12, Journal of Dairy Science. American Dairy Science Association. 102 (2019) 4891-4905).

Although the behaviour of this complex during the treatment by cation exchange chromatography is close to that of lactoferrin, it is possible to vary the vitamin B12 content in the eluate obtained by the method according to the invention depending on the conditions used.

Furthermore, despite its nutritional interest, for certain applications (e.g. infant formulas, i.e. infant formulas/milks and/or follow-on formulas/milks) in certain specific cases (high incorporation dose), there may be an interest in limiting this vitamin B12 content in a pure lactoferrin fraction ingredient. In this context, the method according to the invention, which allows to adjust the final vitamin B12 content, is of great interest.

The invention therefore relates to food products for human or animal consumption comprising an isolate according to the invention; in the context of infant formulas, i.e. infant formulas/milks and/or follow-on formulas/milks, an isolate whose lactoferrin protein purity is >95% and whose vitamin B12 content is 5 μg/g protein is preferably used. The incorporation rate of the isolate according to the invention is 50 to 1000 mg of protein in one litre of a ready-to-eat formula.

The present invention also relates to non-food products, such as hygiene products and cosmetic products comprising an isolate according to the invention.

Also included in the present invention are the products for oral hygiene, such as toothpastes in gel or paste form, mouthwashes, chewing gums, comprising an isolate according to the invention. The incorporation rate of the isolate according to the invention is 1 to 100 mg of protein in one gram of a product.

### FIGURES

FIG. 1: Schematic representation of the method for obtaining the isolate of cationic whey proteins of high purity lactoferrin.

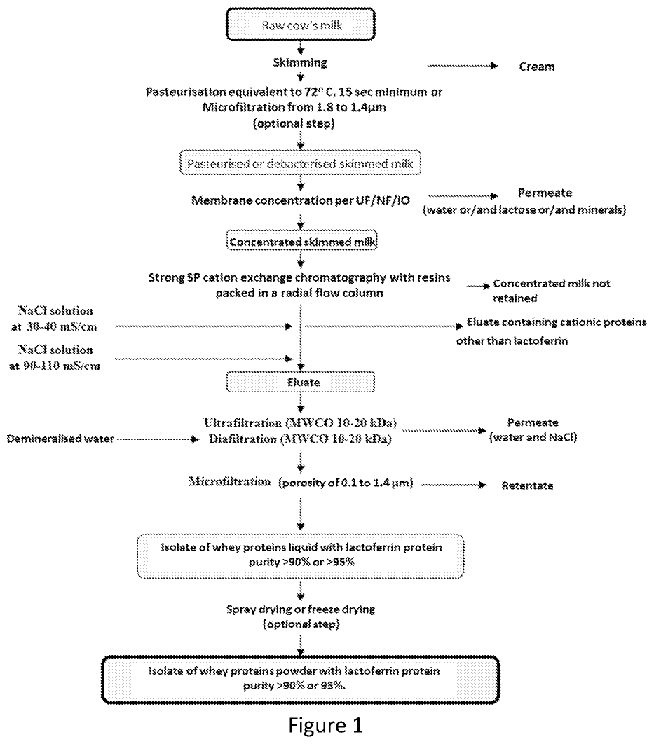

FIG. 2: Pressure loss generated by the radial flow column and different parameters of the pasteurised skimmed milk passage (DM and PM concentrations, DM and PM flow rates) (example 4)

FIG. 3: Correlation between the pressure loss generated by the radial flow column and different parameters of the pasteurised skimmed milk passage (DM and PM flow rates) (example 4)

FIG. 4: RP HPLC profile (Reversed-Phase High-Performance Liquid Chromatography; C18 column 300 Å, 0.1% TFA in H20/CH3CN gradient, detection at 280 nm) of Ingredient 1 of Example 5.

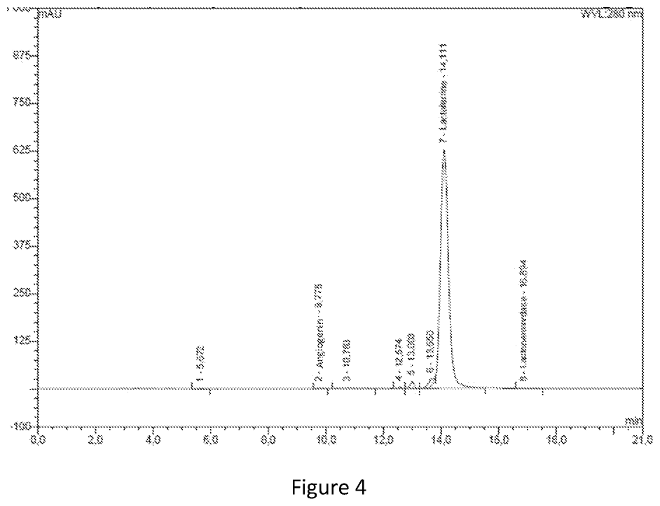

FIG. 5: SEC HPLC profile (Size Exclusion High-Performance Liquid Chromatography; column TSK G3000PWxl, CH3CN/H20/TFA, detection at 210 nm) of Ingredient 1 of Example 5. The molecular weight indications at the top according to the retention times of the control proteins.

FIG. 6: RP HPLC profile of Ingredient 2 of Example 6.

FIG. 7: RP HPLC profile of Ingredient 3 of Example 6.

### Example 1

### Bench Assay with Pasteurised Skimmed Cow's Milk (Control)

1) The skimmed cow's milk whose DM is 92 g/L was pasteurised at 73° C. for 20 seconds, then cooled to 6° C. The concentration of bovine lactoferrin in this pasteurised skimmed milk was measured by HPLC SCX (High Performance Liquid Chromatography by strong cation exchange; column Propac SCX, 20 mM phosphate buffer in NaCl gradient, detection at 280 nm);

2) The pasteurized skimmed milk was passed through an axial flow column (1.6 cm diameter) containing 20 mL (BV) of SP Sepharose Big Beads at a linear speed of 400 cm/hr with a variable volume of pasteurized skimmed milk;

3) After rinsing with 5 BV of osmosis water, the bound proteins are eluted with 6 BV of a 10% (w/v) NaCl solution at 20° C.;

4) The bovine lactoferrin content of each eluate was measured by RP HPLC (Reversed-Phase High-Performance Liquid Chromatography; C18 column 300 Å, 0.1% TFA/CH3CN gradient, detection at 280 nm). Thus, the amount of bovine lactoferrin in each eluate was obtained.

The conditions and the results of the assays are shown in Table 1:

TABLE 1
Bovine lactoferrin obtained with the
passage of skimmed milk at 92 g/L

| Pasteurised skimmed milk | | | | | | Eluate | |
|---|---|---|---|---|---|---|---|
| | | | | Passage | | Lactoferrin | |
| | | | | flow rate | | | (mg/L of |
| DM | Lactoferrin | Volume | | | (g DM/ | | skimmed |
| (g/L) | (mg/L) | (L) | (BV) | (cm/h) | cm2/h) | (mg) | milk) |
| 92 | 89 | 4.00 | 200 | 400 | 36.8 g | 255.5 | 63.9 |
| 92 | 78 | 6.00 | 300 | 400 | 36.8 g | 317.3 | 52.9 |
| 92 | 86 | 9.00 | 450 | 400 | 36.8 g | 511.7 | 56.9 |

### Example 2

### Bench Assay with Concentrated Pasteurised Skimmed Cow's Milk

1) The cow's milk was skimmed, then pasteurised at 73° C. for 20 seconds, then cooled to 6° C. This pasteurised skimmed cow's milk whose DM is 92 g/L was concentrated by a reverse osmosis to 130 g/L DM at 6° C. The concentration of bovine lactoferrin in this concentrated pasteurized skimmed milk was measured by HPLC SCX (column Propac SCX, 20 mM phosphate buffer in NaCl gradient, detection at 280 nm);

2) The concentrated pasteurized skimmed milk (130 g/L DM) is passed through an axial flow column (1.6 cm diameter) containing 20 mL (BV) of SP Sepharose Big Beads at a variable linear speed with variable volume of pasteurized skimmed milk;

3) After rinsing with 5 BV of osmosis water, bound proteins are eluted with 5 BV of a 10% (w/v) NaCl solution at 20° C.;

4) The bovine lactoferrin content of each eluate was measured by RP HPLC (C18 column 300 Å, 0.1% TFA/CH3CN gradient, detection at 280 nm). Thus, the amount of bovine lactoferrin in each eluate was obtained.

The conditions and the results of the assays are presented in Table 2:

TABLE 2
Bovine lactoferrin obtained with the passage
of concentrated skimmed milk at 130 g/L

| Pasteurised skimmed milk | | | | | | Eluate | |
|---|---|---|---|---|---|---|---|
| | | Volume | | Passage | | Lactoferrin | |
| | Lacto- | | (BV) | flow rate | | | (mg/L of |
| DM | ferrin | | [equiv- | | (g DM/ | | skimmed |
| (g/L) | (mg/L) | (L) | alence]* | (cm/h) | cm2/h) | (mg) | milk) |
| 130 | 127 | 4.20 | 210 | 200 | 26.0 | 487.9 | 116.2 |
| | [89] | [5.93] | [297] | | | | [82.2] |
| 130 | 127 | 4.20 | 210 | 300 | 39.0 | 457.8 | 109.0 |
| | [89] | [5.93] | [297] | | | | [77.1] |
| 130 | 127 | 6.00 | 300 | 200 | 26.0 | 671.0 | 118.8 |
| | [89] | [8.48] | [424] | | | | [79.1] |
| 130 | 127 | 6.00 | 300 | 300 | 39.0 | 617.6 | 102.9 |
| | [89] | [8.48] | [424] | | | | [72.8] |

*The equivalent values with the pasteurised non-concentrated skimmed milk (whose DM is 92 g/L).

The comparison of Tables 1 and 2 shows that the yield of bovine lactoferrin obtained is much higher (>20%) with previously concentrated pasteurised skimmed milk using the comparable binding conditions (e.g. the passage of ˜300 BV equivalent of pasteurised skimmed milk with the DM flow rate 37-39 g/cm²/h).

### Example 3

### Bench Assay with the Concentrated Pasteurised Skimmed Cow's Milk

1) The cow's milk was skimmed, then pasteurised at 73° C. for 20 seconds, then cooled to 6° C. This pasteurised skimmed cow's milk whose DM is 92 g/L was concentrated by a reverse osmosis to 130 g/L DM at 6° C. The concentration of bovine lactoferrin in this concentrated pasteurized skimmed milk was measured by HPLC SCX (column Propac SCX, 20 mM phosphate buffer in NaCl gradient, detection at 280 nm);

2) The concentrated pasteurized skimmed milk (130 g/L DM) is passed through an axial flow column (1.6 cm diameter) containing 20 mL (BV) of SP Sepharose Big Beads at a variable linear speed with variable volume of pasteurized skimmed milk;

3) After rinsing with 5 BV of osmosis water, bound proteins are partially eluted with 6 BV of a 2.6% (w/v) NaCl solution at 38 mS/cm at 20° C. Lactoperoxidase, ribonucleases and other basic proteins were recovered from this eluate;

4) Still bound proteins are eluted with 5 BV of a 10% (w/v) NaCl solution at 20° C. The bovine lactoferrin was recovered from this eluate;

5) The proportion of lactoferrin in the total protein in this 2^{nd} eluate was determined by RP HPLC (C18 column 300 Å, 0.1% TEA in H20/CH3CN gradient, detection at 280 nm) as the relative area of the peak of the bovine lactoferrin.

The cobalamin (vitamin B12) content in this 2^{nd} eluate was also measured by the AOAC method. Thus, its total protein content was obtained.

The conditions and the results of 2 series of assays are shown in Table 3:

TABLE 3
Bovine lactoferrin obtained in the 2nd eluate with
the passage of concentrated skimmed milk at 130 g/L

| Pasteurised skimmed milk | | | | | |
|---|---|---|---|---|---|
| | | | Passage | 2^{nd} Eluate | |
| Lactoferrin | Volume | | flow rate | Lactoferrin | Cobalamine |
| (mg/L) | (L) | (BV) | (cm/h) | (%/proteins) | (μg/g proteins) |
| 130 | 1.60 | 80 | 150 | 94.0% | 12.20 |
| 130 | 1.60 | 80 | 200 | 94.3% | 12.15 |
| 130 | 1.60 | 80 | 300 | 94.7% | 9.44 |
| 130 | 1.60 | 80 | 400 | 95.6% | 9.13 |
| 127 | 4.20 | 210 | 200 | 95.2% | 5.53 |
| 127 | 4.20 | 210 | 300 | 95.9% | 2.69 |
| 127 | 6.00 | 300 | 200 | 95.0% | 2.60 |
| 127 | 6.00 | 300 | 300 | 95.4% | 1.85 |

These results show that using appropriate conditions, two kinds of fraction of high lactoferrin purity (e.g. >95% on total proteins) can be obtained by cation exchange chromatography with the passage of concentrated pasteurised skimmed milk:
a fraction whose lactoferrin protein purity is >95% and whose vitamin B12 content is 5 μg/g protein;
a fraction whose lactoferrin protein purity is >90% and whose vitamin B12 content is 10 μg/g protein.

### Example 4

### Industrial Scale Assay with the Pasteurised Skimmed Cow's Milk (Control)

Although the pasteurised skimmed milk concentrated to ˜130 g/L can be passed through an axial column at bench scale, it is difficult to envisage a stable production over time at industrial scale with a passage of a complex matrix such as milk material, especially concentrated, due to both high pressure loss and filter surface clogging.

We checked the pressure loss behaviour through an industrial radial flow column by passing the skimmed milk of different DM and at different flow rates.

1) An industrial radial flow column of 260 L (Albert Handtmann Armaturenfabrick GmbH) was prepared with 280 L of SP Sepharose Big Beads Food Grade resins. It was regenerated with 10% NaCl, then saturated with 1 N NaOH and finally rinsed with osmosis water;

2) The cow's milk was skimmed, then pasteurised at 73° C. for 20 seconds, then cooled to 6° C. A portion of pasteurised skimmed cow's milk was concentrated by a reverse osmosis at 6° C. The compositions of these unconcentrated and concentrated pasteurised skimmed milks are as follows:

TABLE 4
Composition of skimmed milk starters

| | Pasteurised | Skimmed milk |
|---|---|---|
| | skimmed milk | pasteurised by RO |
| DM (g/L) | 87.6 | 231.5 |
| TAM [N × 6.38] (g/L) | 33.7 | 89.2 |
| PM [(N-NPN) × 6.38] (g/L) | 31.9 | 85.4 |

DM: dry material;
TAM: total nitrogenous material;
PM: protein material

3) After preparing the concentration level of pasteurised skimmed milk by in-line mixing, it is passed at different flow rates through the previously prepared radial flow column at a temperature of 10° C.

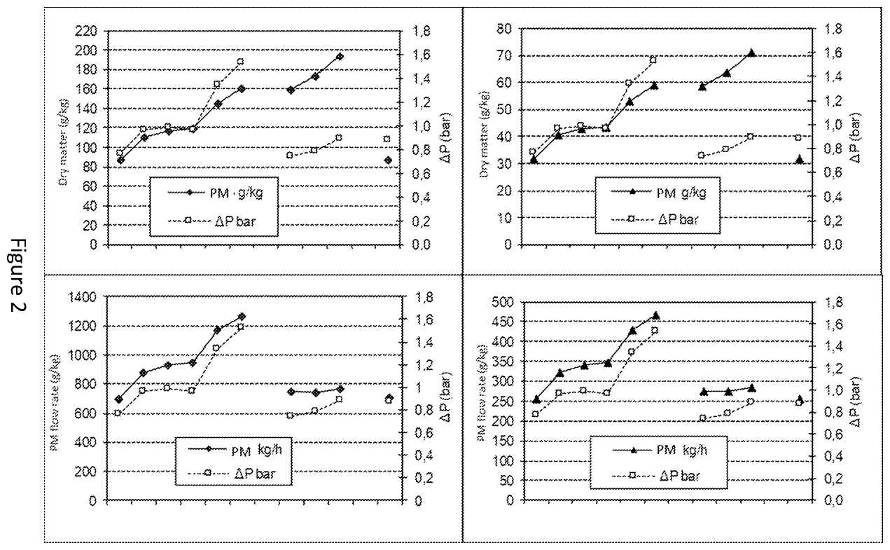

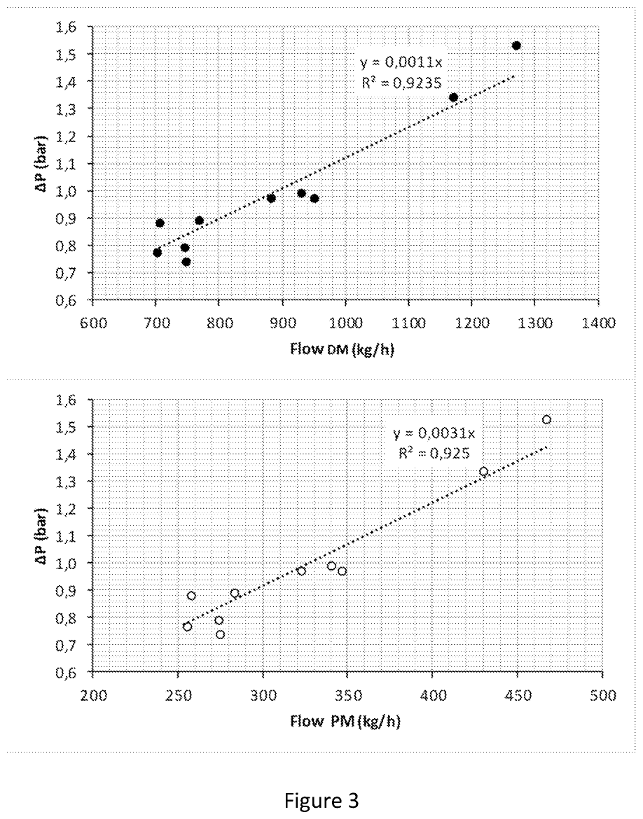

The observed skimmed milk compositions, flow rates and pressures are shown in Table 5. The pressure losses (i.e. pressure) generated by the radial flow column increased as a function of flow rates and mobile phase material concentrations (FIGS. 2) and correlated well with the flow rate in DM or PM (FIG. 3). These results show that this industrial scale radial flow column allows a passage of concentrated pasteurised skimmed milk (by reverse osmosis) up to 200 g/L DM or 72 g PM (or 75 g/L in TAM) with an acceptable pressure loss using an appropriate passage flow rate under routine industrial production conditions.

TABLE 5
The skimmed milk compositions, the flow rates and the pressures observed with the industrial radial flow column

| | | | | | | | | | | | |
|---|---|---|---|---|---|---|---|---|---|---|---|
| Skimmed | DM (g/L) | 87.6 | 110.7 | 116.9 | 119.2 | 145.0 | 160.3 | 159.3 | 173.3 | 193.3 | 87.6 |
| milk | TAM (g/L) | 33.7 | 42.6 | 45.0 | 45.7 | 55.9 | 61.8 | 61.4 | 66.8 | 74.5 | 33.7 |
| composition | PM (g/L) | 31.9 | 40.5 | 42.8 | 43.5 | 53.3 | 58.9 | 58.6 | 63.8 | 71.2 | 31.9 |
| Flow rate | Liquid (m3/h) | 4.1 | 4.0 | 4.0 | 4.0 | 4.1 | 4.0 | 2.4 | 2.2 | 2.0 | 4.1 |
| | DM (kg/h) | 702 | 882 | 929 | 951 | 1170 | 1269 | 748 | 745 | 768 | 707 |
| | PM (kg/h) | 256 | 323 | 340 | 347 | 430 | 467 | 275 | 274 | 283 | 258 |
| Column | Input (bar) | 0.93 | 1.11 | 1.13 | 1.12 | 1.5 | 1.68 | 0.87 | 0.91 | 1.02 | 1.01 |
| pressure | Output (bar) | 0.16 | 0.14 | 0.14 | 0.15 | 0.16 | 0.15 | 0.13 | 0.12 | 0.13 | 0.13 |
| | ΔP (bar) | 0.77 | 0.97 | 0.99 | 0.97 | 1.34 | 1.53 | 0.74 | 0.79 | 0.89 | 0.88 |

### Example 5

### Industrial Assay for the Manufacture of the Isolate of Whey Pure in Bovine Lactoferrin with Concentrated Pasteurised Skimmed Milk

1) The cow's milk was skimmed, then pasteurised at 73° C. for 20 seconds, then cooled to 6° C., then concentrated by a reverse osmosis to 128 g/L DM at 6° C.;

2) 80 m³ of this concentrated pasteurised skimmed milk was passed through an industrial radial flow column of 260 L (Albert Handtmann Armaturenfabrick GmbH) packed with 280 L of SP Sepharose Big Beads Food Grade resins at a flow rate of 2.6 m/h;

3) After rinsing with 5 BV of osmosis water, bound proteins are partially eluted by 6 BV of a 38 mS/cm NaCl solution at 20° C. Lactoperoxidase, ribonucleases and other basic proteins were recovered from this eluate;

4) Still bound proteins are eluted with 4 BV of a 10% (w/v) NaCl solution at 20° C. This eluate containing the bovine lactoferrin was cooled and stored at 6° C.; Steps 2-4 were repeated 10 times;

6) 11.2 m³ of 2nd pooled eluate was concentrated on an ultrafiltration (organic spiral membrane with MWCO of 20 kDa), then diafiltered on UF (MWCO 20 kDa) with osmosis water down to 1 mS/cm, and finally microfiltered on a 1.4 μm ceramic membrane in a double layer (Membrarox®, Pall Corporation);

7) The isolate of whey proteins enriched with bovine lactoferrin obtained in the form of the micro-filtrate has undergone a spray drying and 40 kg of powder was obtained (Ingredient 1);

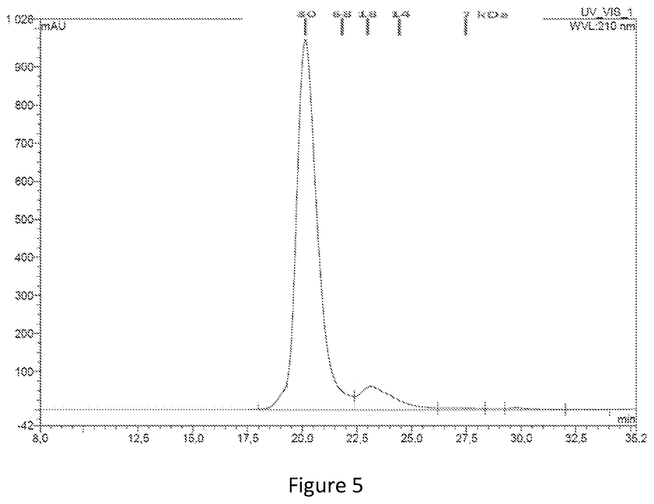

8) The ingredient 1 was analysed; in particular the proportion of lactoferrin in the total protein was determined by RP HPLC (C18 column 300 Å, 0.1% TFA in H20/CH3CN gradient, detection at 280 nm) as the relative peak area of the bovine lactoferrin (FIG. 5). The cobalamin (vitamin B12) content in this 2nd eluate was also measured by the AOAC method. The results of the analyses are presented in Table 6.

### Example 6

### Industrial Assay for the Manufacture of Isolate of Whey Pure in Bovine Lactoferrin with the Concentrated Pasteurised Skimmed Milk

1) The cow's milk was skimmed, then pasteurised at 73° C. for 20 seconds, then cooled to 6° C., then concentrated by a reverse osmosis to 120 g/L DM at 6° C.;

2) 60 m³ of this concentrated pasteurised skimmed milk was passed through a 260 L radial flow industrial column (Albert Handtmann Armaturenfabrick GmbH) packed with 280 L of SP Sepharose Big Beads Food Grade resins at a flow rate of 2.6 m/h;

3) After rinsing with 5 BV of osmosis water, bound proteins are partially eluted by 6 BV of a 36 mS/cm NaCl solution at 20° C. Lactoperoxidase, ribonucleases and other basic proteins were recovered from this eluate;

4) Still bound proteins are eluted with 4 BV of a 10% (w/v) NaCl solution at 20° C.

This eluate containing the bovine lactoferrin was cooled and stored at 6° C.;

5) Steps 2-4 were repeated 15 times;

6) 116.8 m 3 of 2nd pooled eluate were concentrated on an ultrafiltration (organic spiral membrane with cutoff threshold (MWCO) 20 kDa), then diafiltered on UF (MWCO 20 kDa) with osmosis water down to 1 mS/cm, and finally microfiltered on a 0.8 μm ceramic membrane in a double-layer (Membrarox®, Pall Corporation);

7) The isolate of whey proteins enriched with bovine lactoferrin obtained in the form of the micro-filtrate has undergone the following additional treatments to ensure the stability of this following protein fraction:
i. A portion of the micro-filtrate was microfiltered on a 0.2 μm PES membrane (Supor® Pall) and then put into the sterilised 1 L bottles (Ingredient 3)
ii. The remaining micro-filtrate has undergone a spray drying and 60 kg of powder was obtained (Ingredient 2).

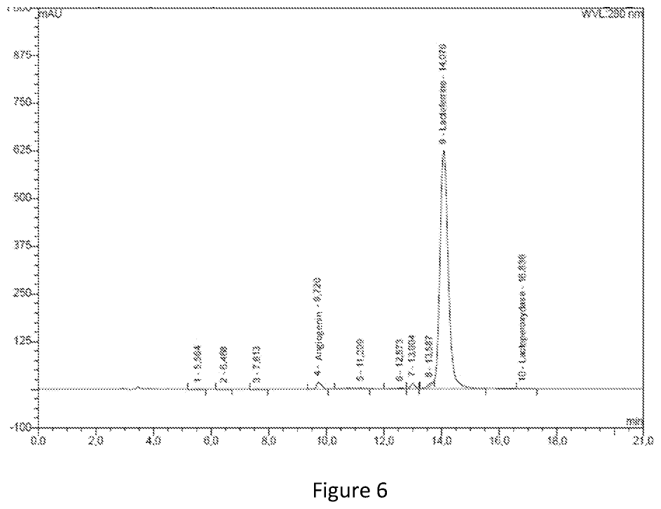

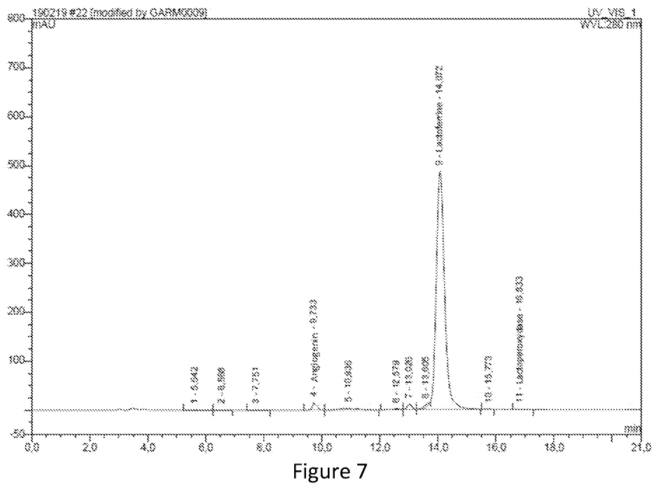

8) The ingredients 2 and 3 were analysed; in particular the proportion of lactoferrin in the total protein in this 2nd eluate was determined by RP HPLC (C18 column 300 Å, 0.1% TFA in H20/CH3CN gradient, detection at 280 nm) as the relative peak area of the bovine lactoferrin (FIG. 6 & FIG. 7). The cobalamin (vitamin B12) content in this 2nd eluate was also measured by the AOAC method. The results of analyses are presented in Table 6.

TABLE 6
The physico-chemical and microbiological characteristics
of Ingredient 1, Ingredient 2 and Ingredient 3

| | Ingredient 1 | Ingredient 2 | Ingredient 3 | |
|---|---|---|---|---|
| Aspect | Fine powder | Fine powder | Liquid | |
| Colour | Light pink | Light pink | Pink | |
| pH | 6.1 | 5.8 | 5.9 | 2% powder solution |
| Solubility (Transmittance | 92 | 93 | 90 | % to 2% powder |
| at 600 nm) | | | | solution |
| Moisture | 3.4 | 3.6 | — | g/100 g |
| Protein (N × 6.38) | 95.9 | 97.2 | 14.1 | g/100 g |
| Ashes | 0.4 | 0.2 | 0.03 | g/100 g |
| Lactoferrin | 95.6 | 94.7 | 94.6 | % protein |
| Vitamine B12 | 2.3 | 8.0 | 8.0 | μg/g protein |
| Mesophilic aerobic flora | <10 | <10 | <1 | UFC/g |
| Coliforms | Absence | Absence | Absence | /g |
| Enterobacteriaceae | Absence | Absence | Absence | /100 g |
| Escherichia coli | Absence | Absence | Absence | /100 g |
| Yeasts and moulds | <10 | <10 | <1 | UFC/g |
| Salmonella | Absence | Absence | Absence | /750 g |
| Positive coagulase | Absence | Absence | Absence | /25 g |
| staphylococci | | | | |
| Listeria | Absence | Absence | Absence | /250 g |
| Cronobacter spp | Absence | Absence | Absence | /750 g |

### Example 7

### Bench Assay with the Concentrated Whey from Pasteurised Skimmed Goat's Milk 1) The goat's milk was skimmed, then pasteurised at 74° C. for 30 seconds, then cooled to 6° C.;

2) 3000 L of pasteurized skimmed goat's milk, after being held at 50° C. for 30 minutes, were passed through a 0.1 μm ceramic microfiltration (Membrarox®, Pall Corporation) to obtain a whey as micro-filtrate of goat's milk free of fat and casein micelles;

3) 2000 L of whey from goat's milk was concentrated on an ultrafiltration (organic spiral membrane with a cutoff threshold (MWCO) of 10 kDa). The compositions of the resulting retentate (450 L) were in Table 7 below:

TABLE 7
Composition of concentrated whey from goat's milk

| | |
|---|---|
| DM (g/L) | 86 |
| TAM [N × 6.38] (g/L) | 33 |
| of which | |
| goat β-Lactoglobuline (g/L) | 21 |
| goat αLactalbumin (g/L) | 10 |
| Goat lactoferrin (g/L) | 0.10 |
| Lactose (g/L) | 38 |
| Ashes (g/L) | 6 |

The concentration of goat β-Lactoglobulin and goat α-Lactalbumin in this concentrated whey was measured by SEC HPLC (TSK G3000PWxl column, CH3CN/H20/TFA, detection at 210 nm). The concentration of goat lactoferrin in this concentrated whey was measured by HPLC SCX (Propac SCX column, 20 mM phosphate buffer in NaCl gradient, detection at 280 nm).

4) 3 L of concentrated goat whey is passed through an axial flow column (1.6 cm diameter) containing 20 mL (BV) of SP Sepharose Big Beads at a linear speed of 200 and 300 cm/h;

After rinsing with 5 BV of osmosis water, bound proteins are partially eluted with 6 BV of a 2.2% (w/v) NaCl solution at 20° C. Cationic proteins other than lactoferrin such as lactoperoxidase were recovered from this eluate;

6) Still bound proteins are eluted with 5 BV of a 10% (w/v) NaCl solution at 20° C. The goat lactoferrin was recovered from this eluate. The goat lactoferrin content in the eluate was measured by RP HPLC (C18 column 300 Å, 0.1% TFA in H20/CH3CN gradient, detection at 280 nm).

As shown in Table 8, a goat lactoferrin fraction whose very high protein purity was extracted very efficiently from concentrated whey from goat's milk.

TABLE 8
Goat lactoferrin obtained in the 2nd eluate with
the passage of concentrated whey from goat's milk

| Concentrated whey from goat's milk | | | 2^{nd} Eluate |
|---|---|---|---|
| | Volume | Passage flow rate | Goat lactoferrin |

| (L) | (BV) | (cm/h) | (%/proteins) | (mg) |
|---|---|---|---|---|
| 3.0 | 150 | 200 | 98.9% | 277 |
| 3.0 | 150 | 300 | 99.0% | 272 |

### Example 8

### Bench Assay with the Concentrated Cheese Whey from Pasteurised Skimmed Goat'S Milk

1) 240 L of cheese whey from pasteurised goat's milk (at 74° C. for 30 seconds) was concentrated on an ultrafiltration (organic spiral membrane with MWCO of 10 kDa). The compositions of the retentate obtained (60 L) were in the table below:

TABLE 9
Composition of the concentrated cheese whey from goat's milk

| | |
|---|---|
| DM (g/L) | 70 |
| TAM [N × 6.38] (g/L) | 36 |
| of which | |
| Goat β-Lactoglobuline (g/L) | 16 |
| Goat α-Lactalbumin (g/L) | 8 |
| Goat lactoferrin (g/L) | 0.26 |
| Lactose (g/L) | 7 |
| Ashes (g/L) | 5 |

The concentration of goat (β-Lactoglobulin and goat α-Lactalbumin in this concentrated whey was measured by SEC HPLC (TSK G3000PWxl column, CH3CN/H20/TFA, detection at 210 nm). The concentration of goat lactoferrin in this concentrated whey was measured by HPLC SCX (Propac SCX column, 20 mM NaPB/NaCl gradient, detection at 280 nm).

2) 3 L of concentrated goat whey is passed through an axial flow column (1.6 cm diameter) containing 20 mL (BV) of SP Sepharose Big Beads at a linear speed of 200 and 300 cm/h;

3) After rinsing with 6 BV of osmosis water, bound proteins are partially eluted with 6 BV of 2.2% (w/v) NaCl solution at 20° C. Cationic proteins other than lactoferrin such as lactoperoxidase were recovered from this eluate;

4) Still bound proteins are eluted with 5 BV of a 10% (w/v) NaCl solution at 20° C. The goat lactoferrin was recovered from this eluate. The goat lactoferrin content in the eluate was measured by RP HPLC (C18 column 300 Å, 0.1% TFA in H20/CH3CN gradient, detection at 280 nm).

As shown in Table 10, a goat lactoferrin fraction with high protein purity was extracted very efficiently from concentrated cheese whey from goat's milk.

TABLE 10
Bovine lactoferrin obtained in the 2^{e} eluate with the
passage of concentrated cheese whey from goat's milk

| Concentrated whey from goat's milk | | | 2^{nd} Eluate |
|---|---|---|---|
| | Volume | Passage flow rate | Goat lactoferrin |

| (L) | (BV) | (cm/h) | (%/proteins) | (mg) |
|---|---|---|---|---|
| 1.2 | 60 | 200 | 93.3% | 293 |
| 1.6 | 80 | 200 | 92.6% | 393 |
| 1.9 | 95 | 200 | 92.3% | 440 |
| 2.4 | 120 | 200 | 90.7% | 506 |

### Example 9

### Preparation Assay of an Infant Milk Powder Supplemented with Bovine Lactoferrin (Low Vitamin B12 Content)

1) An infant milk powder based on cow's milk has been prepared by a standard manufacturing method by formulating with skimmed cow's milk, lactose, maltodextrins, oleic sunflower oil, anhydrous milk fat, demineralised whey, soluble protein, galacto-oligosaccharides, sunflower oil, rapeseed oil, soy lecithin, sunflower lecithin, calcium phosphate, fish oil, potassium phosphate, Mortierella alpina oil, choline bitartrate, calcium chloride, potassium citrate, magnesium citrate, sodium chloride, fructo-oligosaccharides, vitamin C, ferric pyrophosphate, calcium carbonate, taurine, potassium hydroxide, potassium chloride, inositol, nucleotides, L-phenylalanine, tocopherol rich extract, L-ascorbyl palmitate, zinc sulphate, L-tryptophan, vitamin E, potassium iodide, L-carnitine, nicotinamide, sodium selenite, calcium pantothenate, copper sulphate, thiamine, vitamin A, vitamin B6, manganese sulphate, folic acid, vitamin K, biotin, vitamin D, riboflavin, vitamin B12.

2) The infant milk powder has been mixed with Ingredient 1 at an incorporation rate of 82 mg/100 g.

TABLE 11
The composition of an infant milk
powder incorporated in Ingredient 1

| | Units | 100 g | 100 mL |
|---|---|---|---|
| ENERGY VALUE | kcal | 502 | 67 |
| | kJ | 2098 | 282 |
| PROTEIN | g | 9.6 | 1.3 |
| Casein | g | 3.4 | 0.5 |
| Soluble proteins | g | 6.2 | 0.8 |
| Lactoferrin | mg | 74 | 10 |
| FATS | g | 26 | 3.4 |
| Saturated fatty acids | g | 6.7 | 0.9 |
| Linoleic acid | mg | 4500 | 608 |
| α-linolenic acid | mg | 430 | 58 |
| Arachidonic acid (ARA) | mg | 139 | 19 |
| Docosahexaenoic acid (DHA) | mg | 126 | 17 |
| CARBOHYDRATES | g | 56.9 | 7.7 |
| Sugars | g | 37.6 | 5.1 |
| Lactose | g | 37.6 | 5.1 |
| Maltodextrins | g | 19.3 | 2.6 |
| DIETARY FIBRES | g | 3 | 0.4 |
| FOS & GOS | g | 3 | 0.4 |
| MINERAL SALTS | g | | |
| Sodium | mg | 150 | 20 |
| Potassium | mg | 540 | 73 |
| Chloride | mg | 380 | 51 |
| Calcium | mg | 490 | 66 |
| Phosphor | mg | 340 | 46 |
| Magnesium | mg | 42 | 5.7 |
| Iron | mg | 5 | 0.68 |
| Zinc | mg | 3.8 | 0.51 |
| Copper | μg | 400 | 54 |
| Iodine | μg | 100 | 14 |
| Selenium | μg | 21 | 2.8 |
| Manganese | μg | 100 | 14 |
| Fluoride | μg | 275 | 37 |
| VITAMINS | | | |
| Vitamin A (ER) | μg | 430 | 58 |
| Vitamin D | μg | 11 | 1.5 |
| Thiamine | μg | 500 | 68 |
| Riboflavin | μg | 600 | 81 |
| Niacin | mg | 4 | 0.54 |
| Pantothenic acid | mg | 3.2 | 0.43 |
| Vitamin B6 | μg | 400 | 54 |
| Biotin | μg | 16 | 2.2 |
| Folates (EFA) | μg | 175 | 24 |
| Vitamin B12 | μg | 1.1 | 0.14 |
| Vitamin C | mg | 70 | 9.5 |
| Vitamin K | μg | 40 | 5.4 |
| Vitamin E (ET) | mg | 13 | 1.8 |
| Nucleotides | mg | 21 | 2.8 |
| Choline | mg | 160 | 22 |
| Inositol | mg | 53 | 7.2 |
| Taurine | mg | 41 | 5.5 |
| Carnitine | mg | 11 | 1.5 |
| Reconstitution rate | | | 13.5% |

### Example 10

### Assay of Powdered Nutritional Formulation Supplemented in Bovine Lactoferrin (High Vitamin B12 Content)

1) A powder infant milk (food for special medical purposes, i.e. FSMP) based on cow's milk has been prepared by a standard manufacturing method by formulating with skimmed milk, vegetable oils (palm, rapeseed, copra, sunflower), demineralised soluble protein, lactose, starch, locust bean flour, lecithin, calcium citrate, fish oil, Mortierella alpina oil, calcium carbonate, vitamin C, calcium phosphate, potassium citrate, sodium citrate, calcium hydroxide, choline chloride, taurine, vitamin E, inositol, ferrous sulphate, L-tryptophan, potassium chloride, calcium chloride, tocopherol rich extract, L-ascorbyl palmitate, L-carnitine, magnesium sulphate, nucleotides, zinc sulphate, vitamin A, nicotinamide, vitamin K, vitamin D, calcium pantothenate, copper sulphate, thiamine, vitamin B6, riboflavin, manganese sulphate, folic acid, potassium iodide, sodium selenite, biotin.

2) The FSMP infant milk powder has been mixed with Ingredient 2 at an incorporation rate of 400 mg/100 g. An intake of 3.1 μg of vitamin B12 in complex form with transcobalamin per 100 g of the powdered formulation was achieved by this incorporation of Ingredient 2.

TABLE 12
The composition of FSMP powder incorporated into Ingredient 2

| | Units | 100 g | 100 mL |
|---|---|---|---|
| ENERGY VALUE | kcal | 504 | 68 |
| | kJ | 2108 | 284 |
| PROTEIN | g | 11 | 1.5 |
| Casein | g | 6.6 | 0.9 |
| Soluble proteins | g | 4.4 | 0.6 |
| Lactoferrin | mg | 370 | 50 |
| FATS | g | 26 | 3.5 |
| Saturated fatty acids | g | 11.2 | 1.5 |
| Linoleic acid | mg | 4500 | 608 |
| α-linolenic acid | mg | 430 | 58 |
| Arachidonic acid (ARA) | mg | 139 | 19 |
| Docosahexaenoic acid (DHA) | mg | 126 | 17 |
| CARBOHYDRATES | g | 55 | 7.4 |
| Sugars | g | 49.5 | 6.7 |
| Lactose | g | 49.5 | 6.7 |
| Starch | g | 5.5 | 0.7 |
| DIETARY FIBRES | g | 3 | 0.4 |
| MINERAL SALTS | g | | |
| Sodium | mg | 150 | 20 |
| Potassium | mg | 540 | 73 |
| Chloride | mg | 330 | 45 |
| Calcium | mg | 540 | 73 |
| Phosphor | mg | 300 | 41 |
| Magnesium | mg | 42 | 5.7 |
| Iron | mg | 5.0 | 0.68 |
| Zinc | mg | 3.8 | 0.51 |
| Copper | μg | 320 | 43 |
| Iodine | μg | 100 | 14 |
| Selenium | μg | 11 | 1.4 |
| Manganese | μg | 100 | 14 |
| Chrome | μg | 20 | 2.7 |
| Molybdenum | μg | 30 | 4.1 |
| Fluoride | μg | <275 | <37 |
| VITAMINS | | | |
| Vitamin A (ER) | μg | 450 | 58 |
| Vitamin D | μg | 7.5 | 1.0 |
| Thiamine | μg | 500 | 68 |
| Riboflavin | μg | 600 | 81 |
| Niacin | mg | 5.0 | 0.68 |
| Pantothenic acid | mg | 3.2 | 0.43 |
| Vitamin B6 | μg | 400 | 54 |
| Biotin | μg | 16 | 2.2 |
| Folic acid | μg | 70 | 9.5 |
| Vitamin B12 | μg | 3.5 | 0.47 |
| Vitamin C | mg | 70 | 9.5 |
| Vitamin K | μg | 40 | 5.4 |
| Vitamin E (ET) | mg | 10 | 1.4 |
| Nucleotides | mg | 22 | 2.9 |
| Choline | mg | 100 | 14 |
| Inositol | mg | 45 | 6.0 |
| Taurine | mg | 33 | 4.5 |
| Carnitine | mg | 17 | 2.3 |
| Reconstitution rate | | | 13.5% |

### Example 11

### Assay of Powdered Nutritional Formulation Supplemented in Bovine Lactoferrin (High Vitamin B12 Content)

1) A liquid infant milk (foods for special medical purposes, FSMP) based on cow's milk has been prepared by a standard manufacturing method by formulating with Skimmed milk, demineralised soluble protein, vegetable oils (palm, palm kernel, rapeseed, sunflower), lactose, soybean lecithin, sunflower lecithin, sodium citrate, calcium phosphate, potassium citrate, calcium chloride, calcium carbonate, vitamin C, Mortierella alpina oil, fish oil, calcium hydroxide, potassium chloride, vitamin E, choline chloride, taurine, ferrous sulphate, tocopherol-rich extract, L-ascorbyl palmitate, inositol, zinc sulphate, nucleotides, L-carnitine, nicotinamide, vitamin A, magnesium sulphate, vitamin K, vitamin D, calcium pantothenate, copper sulphate, thiamine, vitamin B6, riboflavin, manganese sulphate, folic acid, potassium iodide, sodium selenite, biotin.

2) The FSMP infant milk in liquid form was sterilised by an ultra-high temperature (UHT) thermal treatment and then mixed with Ingredient 3 at an incorporation rate of 410 mg/100 g (on the dry material). An intake of 0.43 μg of vitamin B12 in complex form with transcobalamin per 100 mL of the liquid formulation was achieved by this incorporation of Ingredient 3.

TABLE 13
The composition of FSMP in liquid incorporated into Ingredient 3

| | Units | 100 mL |
|---|---|---|
| ENERGY VALUE | kcal | 69 |
| | kJ | 288 |
| PROTEIN | g | 1.5 |
| Casein | g | 0.6 |
| Soluble proteins | g | 0.9 |
| Lactoferrin | mg | 50 |
| FATS | g | 3.5 |
| Saturated fatty acids | g | 1.6 |
| Linoleic acid | mg | 418 |
| α-linolenic acid | mg | 46 |
| Arachidonic acid (ARA) | mg | 7 |
| Docosahexaenoic acid (DHA) | mg | 7 |
| CARBOHYDRATES | g | 7.8 |
| Sugars | g | 7.8 |
| Lactose | g | 7.8 |
| DIETARY FIBRES | g | 0 |
| MINERAL SALTS | g | |
| Sodium | mg | 20.3 |
| Potassium | mg | 72.9 |
| Chlorure | mg | 44.6 |
| Calcium | mg | 66.2 |
| Phosphore | mg | 36.5 |
| Magnesium | mg | 5.67 |
| Iron | mg | 0.68 |
| Zinc | mg | 0.54 |
| Copper | μg | 43 |
| Iodine | μg | 14 |
| Selenium | μg | 1.4 |
| Manganese | μg | 14 |
| Chrome | μg | 2.7 |
| Molybdenum | μg | 1.4 |
| Fluorure | μg | <37 |
| VITAMINS | | |
| Vitamin A (ER) | μg | 61 |
| Vitamin D | μg | 1.0 |
| Thiamine | μg | 68 |
| Riboflavin | μg | 81 |
| Niacin | mg | 0.54 |
| Pantothenic acid | mg | 0.43 |
| Vitamin B6 | μg | 54 |
| Biotin | μg | 2.2 |
| Folic acid | μg | 9.5 |
| Vitamin B12 | μg | 0.47 |
| Vitamin C | mg | 9.5 |
| Vitamin K | μg | 5.4 |
| Vitamin E (ET) | mg | 2.0 |
| Nucleotides | mg | 2.9 |
| Choline | mg | 14 |
| Inositol | mg | 6.0 |
| Taurine | mg | 4.5 |
| Carnitine | mg | 2.3 |
| Total DM | g | 13.2 |

### Example 12

### Preparation of a Capsule Food Supplement Using the Bovine Lactoferrin (High Vitamin B12 Content)

A capsule food supplement was prepared from the mixture of Ingredient 2 of Example 5 (99.5% of the mixture) and colloidal silica (0.5% of the mixture). Each capsule contains 200 mg of protein.

The content of vitamin B12 in complex form with transcobalamin is 1.6 μg/capsule. The recommended daily dose for each population group is as follows:

TABLE 14

| | | Vitamin B12 | Bovine |
|---|---|---|---|
| Population group | Daily dose | in complex form | lactoferrin |
| Children from 4 to 10 | 1 capsule | 1.6 μg | 189 mg |
| years old | | | |
| Adolescents aged 11 to 17 | 2 capsules | 3.2 μg | 379 mg |
| Adults | 3 capsules | 4.8 μg | 568 mg |
| Pregnant/lactating women | 4 capsules | 5.6 μg | 757 mg |

1. A method for preparing an isolate of cationic whey proteins comprising the following steps a) to f):
a) the starting raw material is a mammalian skimmed milk or a whey from mammalian milk previously concentrated by a membrane technology such that, when the starting raw material is prepared with a mammalian skimmed milk, the concentration of protein material PM is between 40 and 72 g/L and, when the starting raw material is prepared with whey, the concentration of protein material is between 20 and 100 g/L;
b) selectively extracting cationic proteins comprising the following steps:
i. passing the starting raw material through a radial flow chromatography column, containing strong cation exchange resins of the sulphopropyl SP type, preferably with a diameter greater than 100 μm:
the volume of starting raw material, equivalent to the unconcentrated one, is between 40 and 500 times the volume of the resins BV;
the linear speed of the starting raw material passage is between 1.0 and 4.0 m/h;
ii. rinsing with demineralised water:
the volume of demineralised water is between 2 and 6 BV;
the demineralised water passage speed is between 3.0 and 5.0 m/h;
iii. eluting the bound cationic proteins with a saline solution with an electrical conductivity between 30 and 50 mS/cm:
the volume of the saline solution is between 4 and 8 BV;
the passage speed of the saline solution is between 0.3 and 2.0 m/h;
iv. eluting the bound cationic proteins with a saline solution with an electrical conductivity of 80 to 140 mS/cm:
the volume of the saline solution is between 3 and 6 BV;
the passage speed of the saline solution is between 0.5 and 2.5 m/h;
c) concentrating the cationic proteins with high purity lactoferrin eluted in the saline solution using an ultrafiltration membrane with a cutoff threshold between 10 and kDa;
d) demineralizing the cationic proteins with high purity lactoferrin by a diafiltration with demineralised water using an ultrafiltration membrane with a cutoff threshold between 10 and 20 kDa to achieve an ash/protein ratio between 0.001 and 0.03;
e) micro-filtrating with a membrane with a cutoff threshold between 0.2 and 1.4 μm of the concentrated solution of specific cationic proteins to reduce the microbial load;
f) optionally, spray drying or freeze drying the concentrated solution of specific cationic proteins previously microfiltered to obtain a powdered lactoferrin isolate.

2. An isolate of cationic whey proteins obtained by the method according to claim 1, characterised in that the proportion of protein on dry material is greater than or equal to 90% by weight and in that the proportion of lactoferrin on the total proteins is greater than 90% by weight.

3. The isolate of cationic whey proteins from cow's milk or goat's milk obtained by the method according to claim 1, characterised in that the proportion of lactoferrin on the total proteins is higher than 95% by weight and containing a cobalamin in complex form with transcobalamin at a concentration lower than or equal to 5 μg/g of proteins.

4. The isolate of cationic whey proteins from cow's milk or goat's milk obtainable by the method according to claim 1, characterised in that the proportion of lactoferrin on the total proteins is higher than 90% by weight and containing a cobalamin in complex form with transcobalamin at a concentration higher than or equal to 5 μg/g of proteins.

5. The isolate of cationic whey proteins according to claim 4, for its use in the prevention and/or the treatment of a vitamin B12 deficient absorption in patients who have undergone gastrectomy or chronically treated with proton pump inhibitors PPI.

6. A food product for human or animal consumption comprising an isolate according to claim 2.

7. A non-food product comprising an isolate according to claim 2.
